(19) United States
(12) Patent Application Publication
(10) Pub. No.: US 2014/0148647 A1
(43) Pub. Date: May 29, 2014

(54) ENDOSCOPE

(71) Applicant: Olympus Corporation, Tokyo (JP)

(72) Inventor: Yoshiro OKAZAKI, Tokyo (JP)

(73) Assignee: OLYMPUS CORPORATION, Tokyo (JP)

(21) Appl. No.: 14/168,467

(22) Filed: Jan. 30, 2014

Related U.S. Application Data
(63) Continuation of application No. PCT/JP2012/069938, filed on Jul. 31, 2012.

(30) Foreign Application Priority Data
Aug. 3, 2011 (JP) 2011-170305

(51) Int. Cl. A61B 1/01; A61B 1/012

(52) U.S. Cl. CPC A61B 1/01; A61B 1/012
USPC 600/154

(57) ABSTRACT

An endoscope reduces a diameter of an insertion portion, and cleanses an observation optical system with a guide wire being inserted. The endoscope includes: an elongated insertion portion to be inserted into a body cavity; an observation optical system at a distal end of the insertion portion to collect light from a subject; a channel portion formed in a longitudinal direction of the insertion portion, through which a fluid is passed and a guide wire is inserted; a seal portion in the channel portion to seal a gap between the channel portion and the guide wire where the guide wire is inserted through the channel portion; and an opening portion on a proximal end side of the seal portion to discharge a fluid flowing through the channel portion toward the observation optical system.

### CROSS REFERENCE TO RELATED APPLICATIONS

This is a continuation of International Application PCT/JP2012/069938, with an international filing date of Jul. 31, 2012, which is hereby incorporated by reference herein in its entirety. This application claims the benefit of Japanese Patent Application No. 2011-170305, the content of which is incorporated herein by reference.

### TECHNICAL FIELD

The present invention relates to an endoscope.

### BACKGROUND ART

A method has been known of inserting a guide wire into a body cavity, inserting an end of the guide wire placed outside a body from a channel of an endoscope, and inserting the endoscope into the body cavity along the guide wire. According to this method, the guide wire can be easily brought to a back side of an organ, and thus the endoscope can be inserted into the back side of the organ along the guide wire.

When an endoscope is inserted into a pericardial cavity, a body fluid such as blood may adhere to an observation optical system to prevent observation. To solve this problem, conventionally, an endoscope has been known including a cleansing channel (and a cleansing nozzle) for cleansing an observation optical system (for example, see PTL 1). In this endoscope, a balloon that inflates in the cleansing channel prevents dirt from entering the cleansing channel.

### CITATION LIST

### Patent Literature

{PTL 1}
Japanese Unexamined Patent Application, Publication No. Hei5-103752

### SUMMARY OF INVENTION

### Technical Problem

The present invention provides an endoscope that can reduce a diameter of an insertion portion, and cleanse an observation optical system even with a guide wire being inserted.

### Solution to Problem

The present invention adopts solutions as described below.

One aspect of the present invention provides an endoscope including: an elongated insertion portion that can be inserted into a body cavity; an observation optical system that is placed at a distal end of the insertion portion, and collects light from a subject; a channel portion that is formed in a longitudinal direction of the insertion portion, through which a fluid is passed, and through which a guide wire is inserted; a seal portion that is provided in the channel portion, and seals a gap between the channel portion and the guide wire in a state where the guide wire is inserted through the channel portion; and an opening portion that is provided on a proximal end side of the seal portion, and discharges a fluid flowing through the channel portion toward the observation optical system.

### Advantageous Effects of Invention

According to the present invention, a diameter of the insertion portion can be reduced, and the observation optical system can be cleansed with the guide wire being inserted.

### BRIEF DESCRIPTION OF DRAWINGS

FIG. 1 is a general configuration diagram of an endoscope according to a first embodiment of the present invention.

FIG. 2 is a partial enlarged view of a distal end of an insertion portion of the endoscope in FIG. 1.

FIG. 3 is a vertical sectional view of the endoscope in FIG. 1.

FIG. 4 is a partial enlarged view of a seal portion in FIG. 3.

FIG. 5 is a partial enlarged view of a seal portion of an endoscope according to a second embodiment of the present invention.

FIG. 6 is a vertical sectional view of the endoscope in FIG. 5 (when a guide wire is inserted).

FIG. 7 is a vertical sectional view of the endoscope in FIG. 5 (when the guide wire is not inserted).

FIG. 8 is a partial enlarged view of a check valve of an endoscope according to a third embodiment of the present invention.

FIG. 9 is a vertical sectional view of the endoscope in FIG. 8 (when a guide wire is not inserted).

FIG. 10 is a vertical sectional view of the endoscope in FIG. 8 (when the guide wire is inserted).

FIG. 11 is a vertical sectional view of an endoscope according to a fourth embodiment of the present invention (when a guide wire is not inserted).

FIG. 12 is a vertical sectional view of the endoscope in FIG. 11 (when the guide wire is inserted).

FIG. 13 is a vertical sectional view of an endoscope according to a fifth embodiment of the present invention (when a guide wire is inserted).

FIG. 14 is a front view of the endoscope in FIG. 13 from a distal end side of an insertion portion (when the guide wire is inserted).

FIG. 15 is a partial enlarged view of a pressing member in FIG. 13 (when the guide wire is not inserted).

FIG. 16 is a partial enlarged view of the pressing member in FIG. 13 (when the guide wire is inserted).

### Description of Embodiments

### First Embodiment

Now, an endoscope 1 according to a first embodiment of the present invention will be described with reference to the drawings.

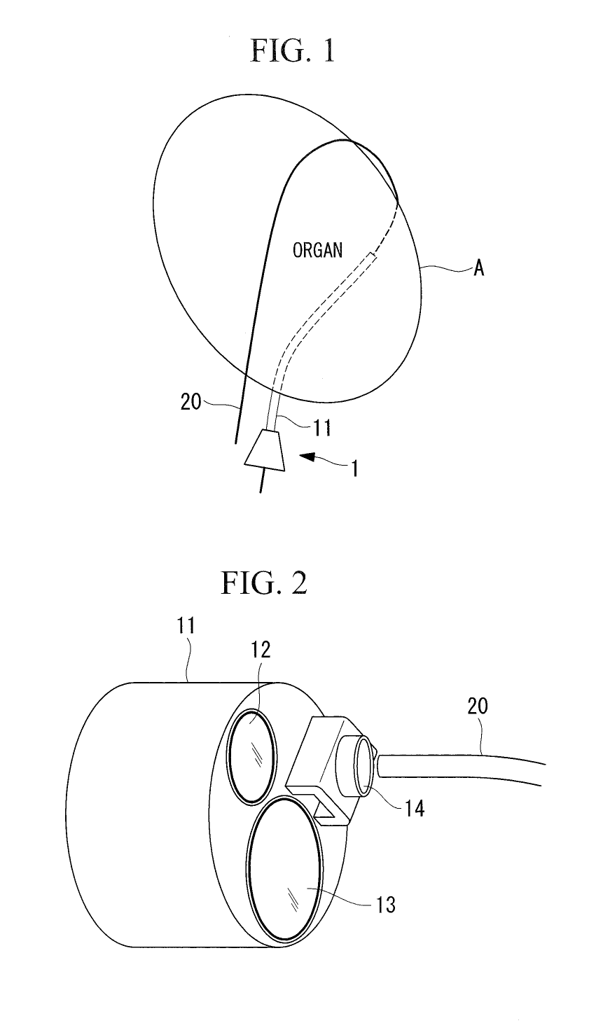

As shown in FIG. 1, the endoscope 1 according to this embodiment includes an elongated insertion portion 11 that can be inserted into a body cavity, for example, a pericardial cavity, and is configured so that the insertion portion 11 is inserted into the body cavity along a guide wire 20.

As shown in FIG. 2, the endoscope 1 according to this embodiment is a direct-view endoscope for observing a front field of view. As shown in FIG. 2, on a distal end surface of the insertion portion 11, an irradiation optical system 12 that applies illumination light to a subject, an observation optical system 13 that collects light from the subject, and a channel portion 14 through which the guide wire 20 is inserted.

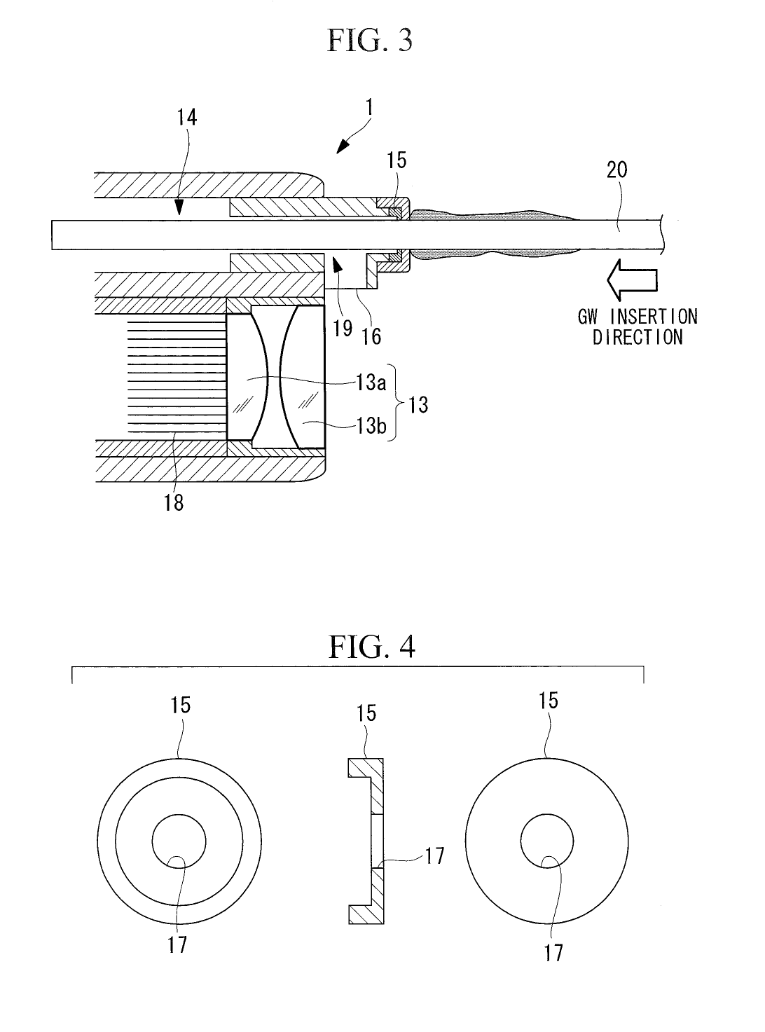

As shown in FIG. 3, the observation optical system 13 includes a pair of lenses 13a and 13b, and on a proximal end side of the lenses, an image guide 18 is placed that guides the light from the subject collected by the lenses 13a and 13b. The image guide 18 is provided in a longitudinal direction in the insertion portion 11, and connected to an image pickup device (not shown) provided on the proximal end side of the insertion portion 11. An image of the subject picked up by the image pickup device is displayed on a display portion (not shown).

In the irradiation optical system 12, similarly, a light guide (not shown) that guides illumination light from an illumination device (not shown) is placed on the proximal end side. The light guide is provided in the longitudinal direction in the insertion portion 11, and connected to the illumination device (not shown) provided on the proximal end side of the insertion portion 11. The illumination light from the illumination device is guided via the light guide to the irradiation optical system 12, and applied to the subject.

As shown in FIG. 3, the channel portion 14 is formed in the longitudinal direction in the insertion portion 11, and a fluid, for example, water is passed through the channel portion 14 and the guide wire 20 is inserted through the channel portion 14. At a distal end of the channel portion 14, a seal portion 15 is provided that seals a gap between the channel portion 14 and the guide wire 20 in a state where the guide wire 20 is inserted through the channel portion 14.

The seal portion 15 is made of an elastic body, for example, rubber, and as shown in FIG. 4, has an insertion hole 17 with an inner diameter substantially equal to an outer diameter of the guide wire 20. With such an insertion hole 17, when the guide wire 20 is inserted through the seal portion 15 (channel portion 14), the seal portion 15 seals the gap between the channel portion 14 and the guide wire 20 to prevent blood or clot adhering to the guide wire 20 from entering the channel portion 14.

As shown in FIG. 3, a branching portion 19 is provided on a proximal end side of the seal portion 15 in the channel portion 14, and an opening portion 16 opening toward the observation optical system 13 is provided in a branching direction of the branching portion 19. With such a configuration, the channel portion 14 discharges the fluid flowing through the channel portion 14 from the opening portion 16 toward the observation optical system 13.

Next, an endoscopic observation method using the endoscope 1 having the above-described configuration will be described. As an example, a case where the endoscope 1 of this embodiment is inserted into a pericardial cavity will be described.

First, the guide wire 20 is inserted into a pericardial cavity A through a sheath (not shown) inserted from a body surface of a patient into the pericardial cavity, and the guide wire 20 is operated under observation on an X-ray transparent image.

Specifically, for example, the sheath is inserted from under the xiphisternum into a body, and passed through different positions of the pericardium near the apex cordis and inserted into the pericardial cavity.

Next, a proximal end portion of the guide wire 20 placed outside a body surface is inserted into the insertion hole 17 in the seal portion 15 provided at a distal end of the insertion portion 11 of the endoscope 1. The guide wire 20 is then inserted through the channel portion 14 in the insertion portion 11. In this state, the insertion portion 11 is inserted along a path of the guide wire 20, and thus the insertion portion 11 of the endoscope 1 is inserted into the pericardial cavity.

In this case, according to the endoscope 1 of this embodiment, the seal portion 15 seals the gap between the channel portion 14 and the guide wire 20 in the state where the guide wire 20 is inserted through the channel portion 14 formed in the longitudinal direction in the insertion portion 11. When the fluid such as water is passed through the channel portion 14 in this state, the fluid flowing through the channel portion 14 is discharged toward the observation optical system 13 from the opening portion 16 provided on the proximal end side of the seal portion 15 in the channel portion 14. Thus, dirt or the like adhering to a surface of the observation optical system 13 can be removed by the fluid, thereby ensuring a satisfactory observation field of view.

Specifically, according to the endoscope 1 of this embodiment, the observation optical system 13 can be cleansed even with the guide wire 20 being inserted through the channel portion 14. Also, the channel portion through which the guide wire 20 is inserted and the channel portion through which the fluid is passed are shared to reduce a diameter of the insertion portion 11, thereby allowing less invasive endoscopic observation.

### Second Embodiment

Next, an endoscope according to a second embodiment of the present invention will be described with reference to the drawings. Hereinafter, for an endoscope in each embodiment, the same matters as in the above-described embodiment are denoted by the same reference numerals and descriptions thereof will be omitted, and different matters will be mainly described.

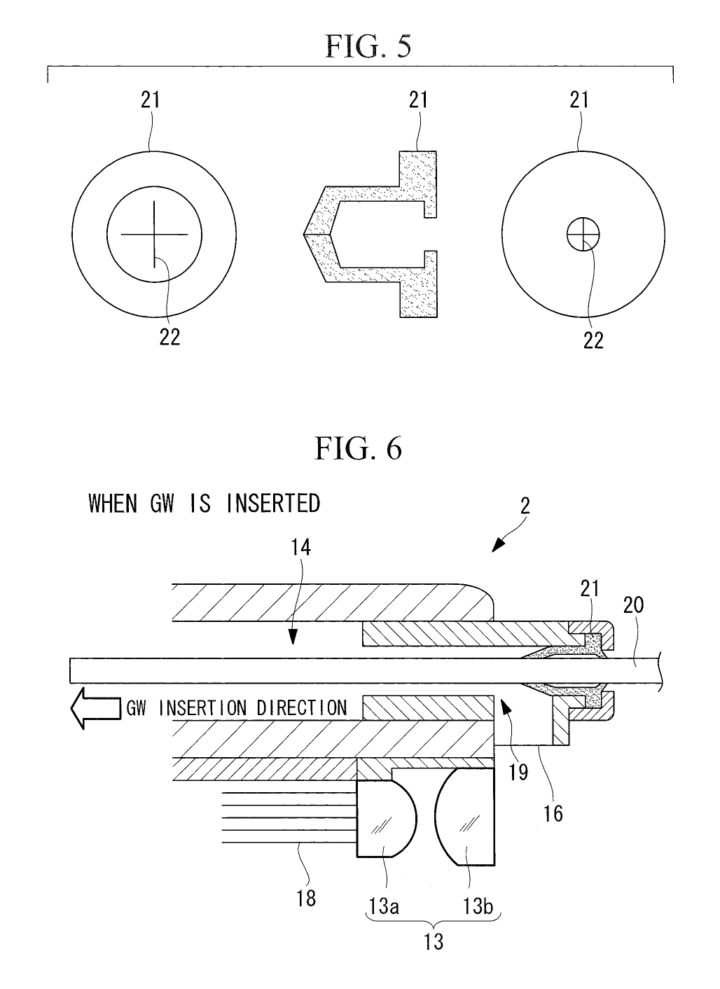

As shown in FIGS. 5 to 7, an endoscope 2 according to this embodiment includes a seal portion (closing portion) 21 having, for example, a cross-shaped cut 22 at a distal end of a channel portion 14, instead of the seal portion 15 having the insertion hole 17 in the endoscope 1 of the first embodiment.

The seal portion 21 is made of an elastic body, for example, rubber, a guide wire 20 is inserted through the cross-shaped cut 22, and the cut is closed in a state where the guide wire 20 is not inserted.

With such a configuration, as shown in FIG. 6, the seal portion 21 seals a gap between the channel portion 14 and the guide wire 20 when the guide wire 20 is inserted through the seal portion 21 (channel portion 14), thereby preventing blood or clot adhering to the guide wire 20 from entering the channel portion 14.

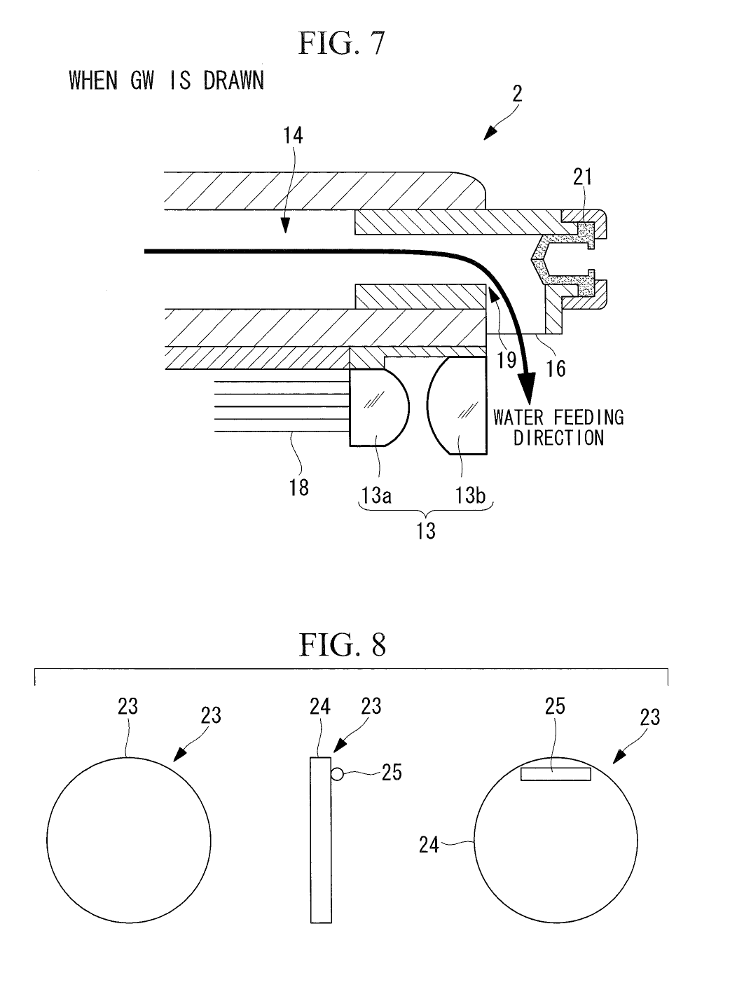

As shown in FIG. 7, the seal portion 21 closes the channel portion 14 in the state where the guide wire 20 is not inserted through the channel portion 14. Thus, a fluid such as water flowing through the channel portion 14 is guided to an opening portion 16.

The seal portion 21 is formed to be recessed with a central portion being closer to a proximal end side so that the guide wire 20 can be easily inserted from a distal end side toward a proximal end side of an insertion portion 11. The cross-shaped cut 22 is provided in the central portion.

According to the endoscope 2 of this embodiment having the above-described configuration, the seal portion 21 can close the channel portion 14 on the distal end side of the opening portion 16 in the state where the guide wire 20 is not inserted through the channel portion 14. Thus, in the state where the guide wire 20 is inserted through the channel portion 14 and also the state where the guide wire 20 is not inserted through the channel portion 14, the fluid such as water flowing through the channel portion 14 can be discharged toward the observation optical system 13 from the opening portion 16 provided in the channel portion 14, and dirt or the like adhering to a surface of the observation optical system 13 can be removed by the fluid.

In the endoscope 2 of this embodiment, the example of the cross-shaped cut 22 being provided in the seal portion 21 has been described. However, the shape of the cut is not limited to this as long as the guide wire 20 can be inserted through the cut, and the cut can close the gap between the guide wire 20 and the channel portion 14 in the state where the guide wire 20 is not inserted.

### Third Embodiment

Next, an endoscope according to a third embodiment of the present invention will be described with reference to the drawings. As shown in FIGS. 8 to 10, an endoscope 3 according to this embodiment includes a check valve (closing portion) 23 that can open and close a channel portion 14, between a seal portion 15 and a branching portion 19 in the channel portion 14.

As shown in FIG. 8, the check valve 23 includes a valve body 24 larger than an inner diameter of the channel portion 14, and a pivot shaft 25 secured to the valve body 24 and provided rotatably in a direction perpendicular to an axis of the channel portion 14. Specifically, the check valve 23 is configured so that the valve body 24 pivots around the pivot shaft 25.

The valve body 24 can pivot to a proximal end side of an insertion portion 11, while being prevented from pivoting to a distal end side of the insertion portion 11 by a wall surface of the channel portion 14. With such a configuration, the check valve 23 allows the guide wire 20 to be inserted from the distal end side to the proximal end side, while preventing a fluid such as water from flowing from the proximal end side to the distal end side.

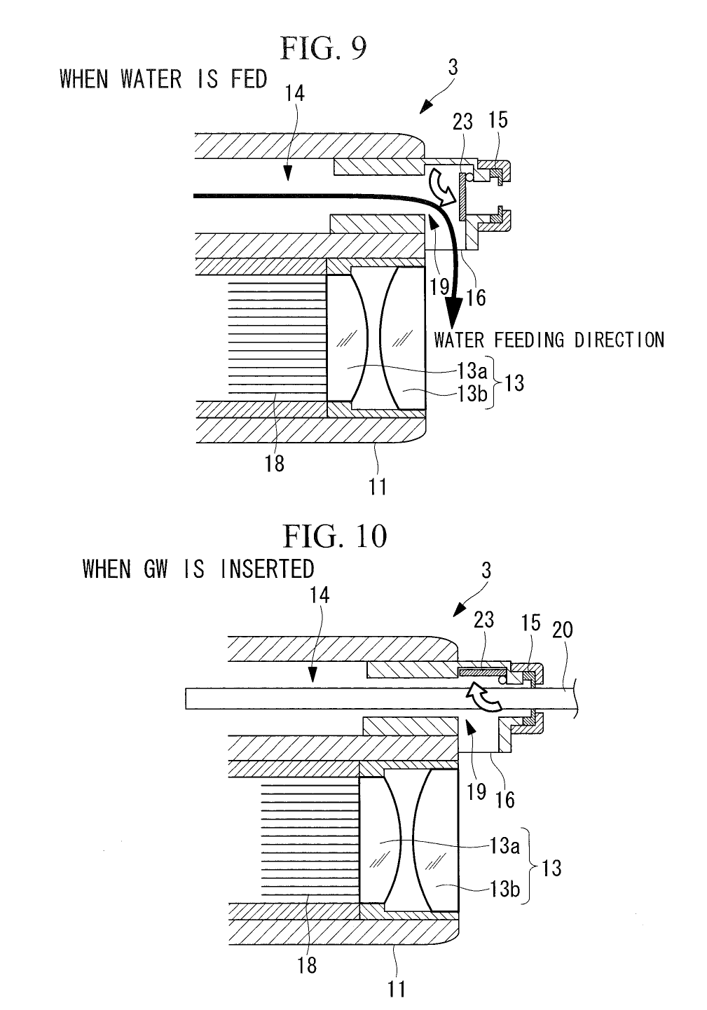

With such a configuration, as shown in FIG. 9, the check valve 23 is urged to the distal end side of the insertion portion 11 by the fluid such as water in the channel portion 14 to close the channel portion 14 in the state where the guide wire 20 is not inserted through the channel portion 14. Thus, the fluid such as water in the channel portion 14 is guided to the opening portion 16.

As shown in FIG. 10, when the guide wire 20 is inserted through the check valve 23 (channel portion 14) from the distal end side to the proximal end side of the insertion portion 11, the valve body 24 pivots to the proximal end side around the pivot shaft 25 to open the channel portion 14.

According to the endoscope 3 of this embodiment having the above-described configuration, the check valve 23 can reliably close the channel portion 14 on the distal end side of the opening portion 16 in the state where the guide wire 20 is not inserted through the channel portion 14. Thus, in the state where the guide wire 20 is inserted through the channel portion 14 and also the state where the guide wire 20 is not inserted through the channel portion 14, the fluid such as water flowing through the channel portion 14 can be discharged toward the observation optical system 13 from the opening portion 16 provided in the channel portion 14, and dirt or the like adhering to a surface of the observation optical system 13 can be removed by the fluid.

### Fourth Embodiment

Next, an endoscope according to a fourth embodiment of the present invention will be described with reference to the drawings.

An endoscope 4 according to this embodiment is a side viewing endoscope including an image pickup device on an outer peripheral surface of an insertion portion 11. For the endoscope 4 of this embodiment, an example will be described in which a seal portion (closing portion) 21 is provided at a distal end of a channel portion 14 as in the endoscope 2 of the second embodiment.

The endoscope 4 of this embodiment includes an LED 31 placed with an optical axis being directed radially outward of the insertion portion 11, an irradiation optical system 32 placed on the optical axis of the LED 31 on an outer peripheral surface of the insertion portion 11, an image pickup device 33 placed with an optical axis being directed radially outward of the insertion portion 11, an observation optical system 34 placed on the optical axis of the image pickup device 33 on the outer peripheral surface of the insertion portion 11, and a nozzle 35 that discharges a fluid flowing through the channel portion 14 toward the observation optical system 34. The LED 31 and the image pickup device 33 are placed close to each other in a longitudinal direction of the insertion portion 11.

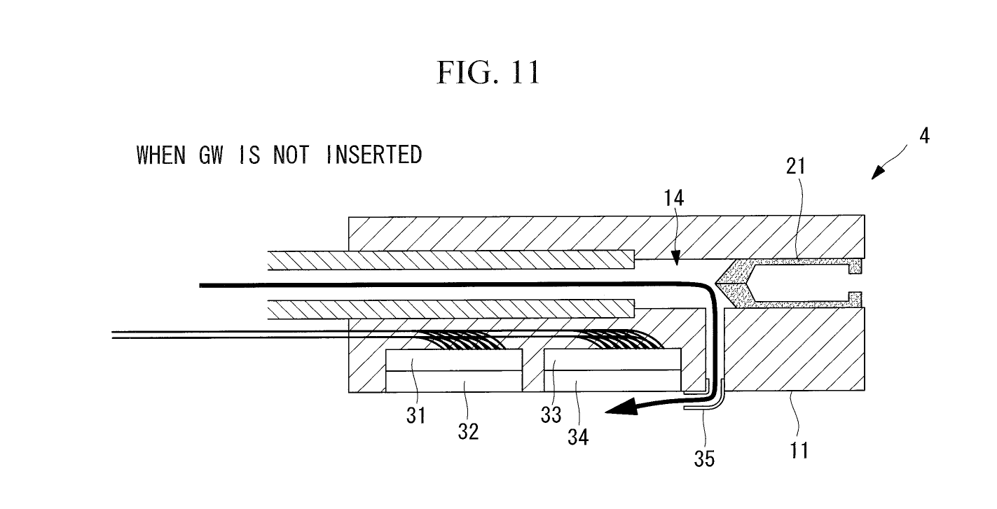

According to the endoscope 4 of this embodiment, as shown in FIG. 11, the seal portion 21 closes the channel portion 14 in a state where the guide wire 20 is not inserted through the channel portion 14. Thus, the fluid such as water in the channel portion 14 can be guided to the nozzle 35 and discharged toward the observation optical system 34.

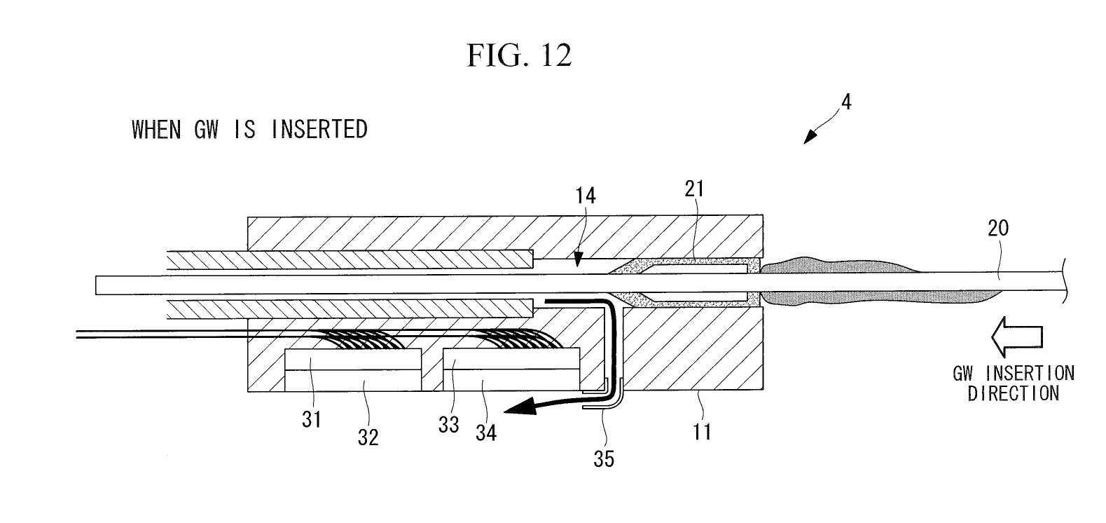

As shown in FIG. 12, when the guide wire 20 is inserted through the seal portion 21 (channel portion 14), the seal portion 21 seals a gap between the channel portion 14 and the guide wire 20 to prevent blood or clot adhering to the guide wire 20 from entering the channel portion 14. Further, the fluid such as water in the channel portion 14 is guided to the nozzle 35 and discharged toward the observation optical system 34.

According to the endoscope 4 of this embodiment having the above-described configuration, the seal portion 21 can close the channel portion 14 on the distal end side of the opening portion 16 in the state where the guide wire 20 is not inserted through the channel portion 14. Thus, in the state where the guide wire 20 is inserted through the channel portion 14 and also the state where the guide wire 20 is not inserted through the channel portion 14, the fluid such as water flowing through the channel portion 14 can be discharged from the nozzle 35 toward the observation optical system 34, and dirt or the like adhering to a surface of the observation optical system 34 can be removed by the fluid.

### Fifth Embodiment

Next, an endoscope according to a fifth embodiment of the present invention will be described with reference to the drawings.

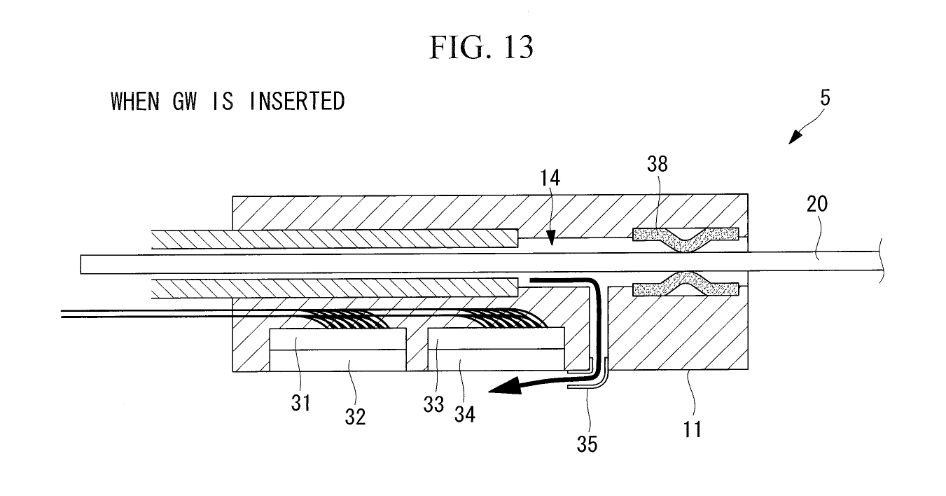

As shown in FIGS. 13 to 16, an endoscope 5 according to this embodiment includes a plurality of pressing members (closing portion) 38 to be pressed radially inward of a channel portion 14 at a distal end of the channel portion 14, instead of the seal portion 15 in the endoscope 1 of the first embodiment.

As shown in FIGS. 15 and 16, the pressing member 38 includes a pair of pressing members 38a and 38b made of an elastic body, for example, rubber. As shown in FIG. 15, the pressing member 38 is formed so that the pair of pressing members 38a and 38b inflate radially inward of the insertion portion 11 when a guide wire 20 is not inserted. Thus, the pressing member 38 closes the channel portion 14 in a state where the guide wire 20 is not inserted.

With such a configuration, as shown in FIG. 15, the pressing member 38 closes the channel portion 14 in the state where the guide wire 20 is not inserted through the channel portion 14. Thus, a fluid such as water in the channel portion 14 is guided to an opening portion 16.

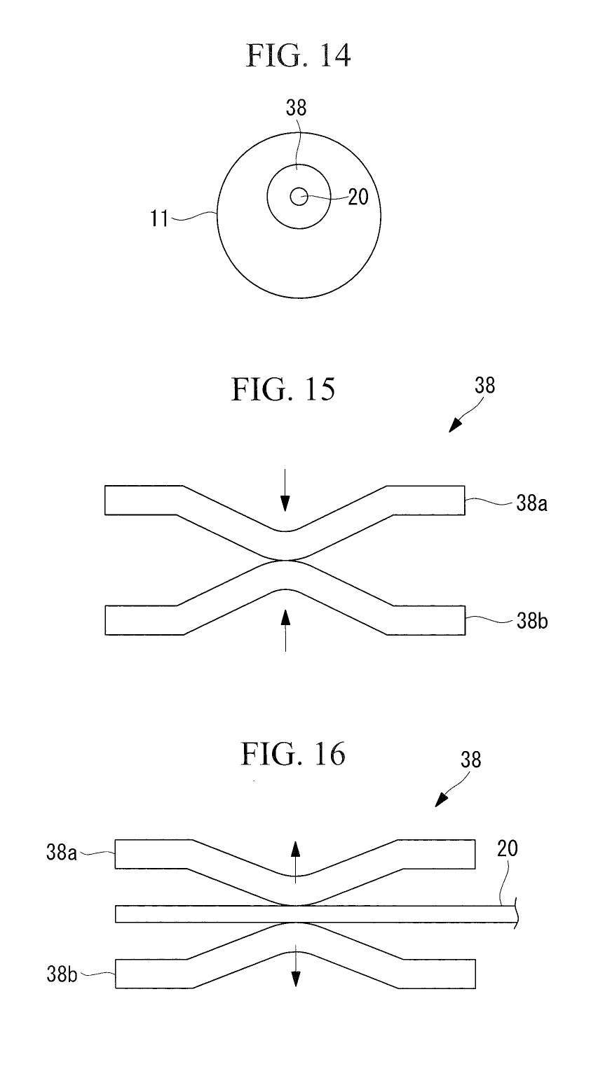

As shown in FIG. 16, when the guide wire 20 is inserted, the pressing member 38 is deformed so that the pair of pressing members 38a and 38b inflate radially outward by an outer diameter of the guide wire 20. Thus, as shown in FIG. 14, the pressing member 38 seals a gap between the channel portion 14 and the guide wire 20 in a state where the guide wire 20 is inserted.

With such a configuration, as shown in FIG. 16, when the guide wire 20 is inserted through the pressing member 38 (channel portion 14), the pressing member 38 seals the gap between the channel portion 14 and the guide wire 20 to prevent blood or clot adhering to the guide wire 20 from entering the channel portion 14.

According to the endoscope 5 of this embodiment having the above-described configuration, the pressing member 38 can close the channel portion 14 on the distal end side of the opening portion 16 with a simple configuration in the state where the guide wire 20 is not inserted through the channel portion 14. Thus, in the state where the guide wire 20 is inserted through the channel portion 14 and also the state where the guide wire 20 is not inserted through the channel portion 14, the fluid such as water flowing through the channel portion 14 can be discharged from the nozzle 35 toward the observation optical system 34, and dirt or the like adhering to a surface of the observation optical system 34 can be removed by the fluid.

For the endoscope 5 of this embodiment, the example of the pressing member 38 including the pair of pressing members 38a and 38b has been described, but not limited to this, the pressing member 38 may include three or more pressing members.

The embodiments of the present invention have been described in detail with reference to the drawings. Specific configurations are not limited to the embodiments, but include matters of design choice without departing from the gist of the present invention. For example, the present invention may be applied to an embodiment that is a combination of the above-described embodiments.

### REFERENCE SIGNS LIST

1, 2, 3, 4, 5 endoscope
11 insertion portion
12 irradiation optical system
13 observation optical system
14 channel portion
15 seal portion
16 opening portion
19 branching portion
20 guide wire
21 seal portion (closing portion)
23 check valve (closing portion)
31 LED
32 irradiation optical system
33 image pickup device
34 observation optical system
35 nozzle
38 pressing member (closing portion)

1. An endoscope comprising:
an elongated insertion portion that can be inserted into a body cavity;
an observation optical system that is placed at a distal end of the insertion portion, and collects light from a subject;
a channel portion that is formed in a longitudinal direction of the insertion portion, through which a fluid is passed, and through which a guide wire is inserted;
a seal portion that is provided in the channel portion, and seals a gap between the channel portion and the guide wire in a state where the guide wire is inserted through the channel portion; and
an opening portion that is provided on a proximal end side of the seal portion, and discharges a fluid flowing through the channel portion toward the observation optical system.

2. The endoscope according to claim 1, further comprising a closing portion that is provided on a distal end side of the opening portion, and closes the channel portion in a state where the guide wire is not inserted through the channel portion.

3. The endoscope according to claim 2, wherein the closing portion is a check valve that allows the guide wire to be inserted from the distal end side to the proximal end side, while preventing the fluid from flowing from the proximal end side to the distal end side.

4. The endoscope according to claim 2, wherein the closing portion is an elastic body having an insertion hole that expands radially outward when the guide wire is inserted, while contracting radially inward when the guide wire is drawn.

5. The endoscope according to claim 2, wherein the closing portion is a plurality of pressing members to be pressed radially inward of the channel portion.
